(19) United States
(12) Patent Application Publication
(10) Pub. No.: US 2005/0118244 A1
(43) Pub. Date: Jun. 2, 2005

(54) Transmittal therapeutic systems containing steroid hormones and propylene glycol monocaprylate

(75) Inventors: Frank Theobald, Bad Breisig (DE); Rene Eifler Rubenacher, Mulheim-Karlich (DE)

Correspondence Address: William F Lawrence;Frommer Lawrence & Haug, 745 Fifth Avenue, New York, NY, 10151, US

(73) Assignee: LTS Lohmann Therapie-System AG, Andernach (DE)

(21) Appl. No.: 10/497,057

(22) PCT Filed: Nov. 16, 2002
(86) PCT No.: PCT/EP02/12873

(30) Foreign Application Priority Data
Dec. 1, 2001 (DE) 101 59 120.9

(51) Int. Cl. A61K 31/56; A61K 9/70; A61K 31/57

(52) U.S. Cl. 424/449; 514/170

(57) ABSTRACT

The invention relates to transdermal therapeutic systems containing at least one steroid hormone and propylene glycol monocaprylate as permeation enhancers. The invention also relates to methods for the production of said systems and to the use of the same in hormone substitution therapy and contraception.

Transdermal therapeutic systems (TTS) are pharmaceutical dosage forms which have a layered structure and have an active ingredient-impermeable backing layer, an active ingredient-containing reservoir layer and an adhesive layer which ensures firm contact to the surface of a user's skin. There is the type of a monolithic matrix system in which the active ingredient-containing reservoir layer and the adhesive layer are identical; the reservoir type in which an additional rate-controlling membrane is disposed between active ingredient-containing reservoir layer and adhesive layer; the type of the multilayer matrix system which has at least two active ingredient-containing reservoir layers, and microreservoir-containing TTS types. It is common to all that they make continuous supply of active ingredient possible over the entire administration period. Their concentration and time profiles therefore resemble those with continuous drip infusions. Numerous transdermal therapeutic systems with various active ingredients are currently available on the pharmaceuticals market.

One of the most important areas of indication for transdermal therapeutic systems is hormone replacement therapy. In particular, postmenopausal hormone replacement can advantageously be effected by means of transdermal therapeutic systems. While in the initial period in particular estrogen-containing single-component products were employed for postmenopausal hormone replacement, the trend now is toward combination of estrogen- and progesterone-containing transdermal combination patches. Such active ingredient combinations can likewise be employed for contraception by means of transdermal therapeutic systems. Testosterone, the male sex hormone, is likewise among the steroid hormones used in hormone replacement therapy (e.g. for the treatment of hypogonadism).

To achieve the necessary plasma levels for the abovementioned indications there is frequently a need for so-called penetration promoters (permeation enhancers). These increase the transport of active ingredient from the transdermal therapeutic system into the blood stream. In addition, they improve the active ingredient utilization of the transdermal therapeutic system, which is desired and worthwhile also for pharma-economic reasons. This means that the same therapeutic effect is possible with a smaller active ingredient loading of the transdermal therapeutic system. The advantage for the patients of the use of such penetration promoters is that the area of application of the transdermal therapeutic system is reduced and thus user compliance can be improved.

U.S. Pat. No. 5,122,383 discloses the use of sorbitan esters, and U.S. Pat. No. 4,863,738 discloses the use of glycerol monooleate, in each case as penetration promoter on use of steroids in transdermal therapeutic systems.

EP-A-279 977 describes the use of propylene glycol laurate and propylene glycol dipelarginate as penetration promoters for transdermal administration of sex hormones (progesterone and estradiol).

EP-A-272 987 discloses the use of mono- and difatty acid esters of propylene glycol, especially of propylene glycol mono- and dilaurate, propylene glycol monopalmitate, propylene glycol monostearate and propylene glycol monooleate as percutaneous absorption enhancers in transdermal therapeutic systems, where the active ingredients may be inter alia steroids and fentanyl or fentanyl derivatives. It has now been found, surprisingly, that on use of a propylene glycol monofatty acid ester which has not been mentioned in the prior art in the list of numerous propylene glycol fatty acid esters to be used as penetration promoters, namely propylene glycol monocaprylate, as penetration promoter in steroid hormone-containing transdermal systems an unexpectedly high flux of active ingredient is achieved, especially with progestogens and testosterone. Steroid hormones which are suitable according to the invention are the following: estradiol, ethinylestradiol, as progestogens progesterone, medroxyprogesterone, hydroxyprogesterone, levonorgestrel, norethisterone acetate, norgestrel, lynestrenol, ethynodiol diacetate, allylestrenol, the progestogen (17α)-17-hydroxy-11-methylene-19-norpregna-4,15-dien-20-yn-3-one (with the name Org 30659, supplied by Organon, Oss, NL) and others, as male hormone testosterone (this list not being intended to be regarded as restrictive).

Examples 1 and 2 below show that on use of 5% propylene glycol monocaprylate it is possible to increase the flux of active ingredient by about 15% (progestogens) and by about 20% (testosterone).

The measurements were carried out as follows: a modified Franz diffusion cell and, in each case, a transdermal therapeutic system with an effective diffusion area of 4.1 cm² was used. The test temperature was 32° C. The flux of active ingredient was measured on an epidermis from human skin. The acceptor used was an aqueous solution of 0.1% hydroxypropyl-β-cyclodextrin+0.1% NaN₃; the acceptor volume was 9 ml (volume exchange after 32, 48, 56 and 72 hours).

### EXAMPLE 1

| | | Cumulative |
|---|---|---|
| | | amount of active |
| | | ingredient |
| | % by | [μg/cm²] after 3 |
| Formula | weight | days |
| Progestogen (Org 30659) | 5.0 | 44.7 |
| Durotak 2287 | 95.0 | |
| Progestogen (Org 30659) | 5.0 | 51.3 |
| Durotak 2287 | 90.0 | |
| Propylene glycol monocaprylate | 5.0 | |

### EXAMPLE 2

| | | Cumulative |
|---|---|---|
| | | amount of active |
| | | ingredient |
| | % by | [μg/cm²] after 3 |
| Formula | weight | days |
| Testosterone | 2.0 | 30.2 |
| Durotak 2287 | 98.0 | |
| Testosterone | 2.0 | 36.2 |
| Durotak 2287 | 93.0 | |
| Propylene glycol monocaprylate | 5.0 | |

1. A transdermal therapeutic system comprising at least one steroid hormone and propylene glycol monocaprylate as penetration promoter.

2. The transdermal therapeutic system as claimed in claim 1, characterized in that estradiol, ethinylestradiol, progesterone, medroxy-progesterone, hydroxyprogesterone, levonorgestrel, (17α)-17-hydroxy-11-methylene-19-norpregna-4,15-dien-20-yn-3-one (Org 30659), norethisterone acetate, norgestrel, lynestrenol, allylestrenol, ethynodiol diacetate or testosterone is used as steroid hormone.

3. The transdermal therapeutic system as claimed in claim 1 or 2, characterized in that it comprises a combination of the active ingredients estradiol or ethinylestradiol with a progestogen.

4. The transdermal therapeutic system as claimed in claim 3, characterized in that it comprises the compound with the name Org 30659 as progestogen.

5. The transdermal therapeutic system as claimed in one or more of the preceding claims, characterized in that the active ingredient-containing matrix consists of a polymer of the group of polyacrylate, polydimethylsiloxane, polyisobutylene, polystyrene, styrene-isoprene-styrene block copolymer, styrene-butadiene-styrene block copolymer, resins, ethylvinyl acetate or of a combination of at least two of these polymers.

6. The transdermal therapeutic system as claimed in one or more of the preceding claims, characterized in that the content of propylene glycol monocaprylate is between 1 and 20% by weight, preferably between 2 and 10% by weight.

7. The transdermal therapeutic system as claimed in one or more of the preceding claims, characterized in that it is intended for hormone replacement therapy.

8. The transdermal therapeutic system as claimed in one or more of claims 1 to 6, characterized in that it is intended for contraception.

9. The use of propylene glycol monocaprylate for increasing the transdermal transport of a steroid hormone active ingredient.

10. The use as claimed in claim 9, characterized in that the relevant steroid hormone is selected from the group comprising estradiol, ethinylestradiol, progesterone, medroxyyprogesterone, hydroxy-progesterone, levonorgestrel, (17α)-17-hydroxy-11-methylene-19-norpregna-4,15-dien-20-yn-3-one (Org 30659), noresthisterone acetate, norgestrel, lynestrenol, allylestrenol, ethynodiol diacetate and testosterone.
